SEQUENCE LISTING (10 sequences)

SEQ ID NO: 1 - DNA, 282 bp, synthetic construct
  source 1..282: mol_type = other DNA
cgggatcctaatgaccaaggaaagcatgattcttcacaccgagttcatcccttatgtgatggaccctatacgcggccgccctggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgtgcatgtattgaacagcgaccttgccggtgccagtcggatagtgttccgagctccc

SEQ ID NO: 2 - DNA, 147 bp, synthetic construct
  source 1..147: mol_type = other DNA
gacctcaagagtaataatatatttcttcatgaagacctcacagtaaaaataggtgattttggtctagctacagagaaatctcgatggagtgggtcccatcagtttgaacagttgtctggatccattttgtggatggcaccagaagtc

SEQ ID NO: 3 - DNA, 147 bp, synthetic construct
  source 1..147: mol_type = other DNA
atggccagcgtggacaacccccacgtgtgccgcctgctgggcatctgcctcacctccaccgtgcaactcatcatgcagctcatgcccttcggctgcctcctggactatgtccgggaacacaaagacaatattggctcccagtacctg

SEQ ID NO: 4 - DNA, 147 bp, synthetic construct
  source 1..147: mol_type = other DNA
atgactgaatataaacttgtggtagttggagctgatggcgtaggcaagagtgccttgacgatacagctaattcagaatcattttgtggacgaatatgatccaacaatagaggattcctacaggaagcaagtagtaattgatggagaa

SEQ ID NO: 5 - DNA, 36 bp, synthetic construct
  source 1..36: mol_type = other DNA
gacctcaagagtaataatatatttcttcatgaagac

SEQ ID NO: 6 - DNA, 19 bp, synthetic construct
  source 1..19: mol_type = other DNA
gacttctggtgccatccac

SEQ ID NO: 7 - DNA, 18 bp, synthetic construct
  source 1..18: mol_type = other DNA
atggccagcgtggacaac

SEQ ID NO: 8 - DNA, 28 bp, synthetic construct
  source 1..28: mol_type = other DNA
caggtactgggagccaatattgtctttg

SEQ ID NO: 9 - DNA, 30 bp, synthetic construct
  source 1..30: mol_type = other DNA
atgactgaatataaacttgtggtagttgga

SEQ ID NO: 10 - DNA, 27 bp, synthetic construct
  source 1..27: mol_type = other DNA
ttctccatcaattactacttgcttcct

(19) United States
(12) Patent Application Publication
(10) Pub. No.: US 2026/0242864 A1
(43) Pub. Date: Aug. 20, 2026

(54) RECOMBINANT NUCLEOSOME MATERIALS

(71) Applicant: Belgian Volition SRL, Isnes (BE)

(72) Inventors: Jacob Vincent MICALLEF, Isnes (BE); Theresa K. KELLY, Isnes (BE); Mhammed BOUGOLISSA, Isnes (BE); Annalisa CANALE, isnes (BE); Priscilla VAN DEN ACKERVEKEN, Isnes (BE); Mark Edward ECCLESTON, Isnes (BE)

(21) Appl. No.: 19/471,972

(22) PCT Filed: Apr. 3, 2024
(86) PCT No.: PCT/EP2024/059081

§ 371 Date: Oct. 2, 2025

Related U.S. Application Data
(60) Provisional application No. 63/493,978, filed on Apr. 3, 2023.

(51) Int. Cl. C12Q 1/6869; C12Q 1/6806; C12Q 1/6886; C40B 40/06; C40B 50/06; C40B 70/00

(52) U.S. Cl. CPC C12Q 1/6869; C12Q 1/6806; C12Q 1/6886; C40B 40/06; C40B 50/06; C40B 70/00; C12Q 2600/118; C12Q 2600/156; C12Q 2600/166

(57) ABSTRACT

The invention relates to methods for assessing the performance of a DNA sequencing assay by using recombinant nucleosomes comprising a target endogenous nucleotide sequence as the control material. The invention also relates to recombinant nucleosomes which, for example, may be used in methods of the invention.

### FIELD OF THE INVENTION

The invention relates to recombinant nucleosomes comprising endogenous sequence DNA fragments and applications thereof, particularly for standardising sequencing assays.

### BACKGROUND OF THE INVENTION

The DNA of eukaryotic organisms is packaged as chromatin such that it can be contained within the nucleus and facilitate epigenetic regulation. The human genome, for example, comprises some 30 billion base pairs, or approximately 2 metres, of DNA packed into a cell nucleus of 10-100 μm diameter whilst also facilitating the control of the cell specific pattern of gene expression characteristic of some 200 different cell types within the body.

The nucleosome is the basic repeating unit of chromatin structure. Nucleosomes play a key role in dictating the accessibility of the eukaryotic genome and are involved in the regulation of DNA transcription, replication and repair. A nucleosome consists of a protein complex of eight highly conserved core histones (comprising of a pair of each of the histones H2A, H2B, H3, and H4). Around this complex is wrapped approximately 147 base pairs (bp) of DNA. Another histone, H1 or H5, acts as a linker and is involved in chromatin compaction. The DNA is wound around consecutive nucleosomes connected by additional linker DNA, or regions of nucleosome depletion, in a structure often said to resemble “beads on a string” and this forms the basic structure of open or euchromatin. In compacted or heterochromatin this string is coiled and super coiled into a closed and complex structure (Herranz and Esteller; 2007).

Recombinant nucleosomes have been synthesised to study their dynamics and for use as enzyme substrates, including for epigenetic regulators, and as calibration materials for immunochemical assays (see for example, Xiao et al; 2005 and Van den Ackerveken et al; 2021). Methods for the assembly of histones H2A, H2B, H3, H4 and DNA to form recombinant nucleosomes are known in the art. Typically, equimolar amounts of purified histones H2A, H2B, H3 and H4 are assembled to form a core histone octamer. A fragment of DNA is then bound around the histone octamer to form a nucleosome.

For current applications of recombinant nucleosomes, the nucleotide sequence of the DNA contained within the nucleosome is not critical. For this reason, most laboratories use a non-natural DNA sequence referred to as the Widom 601 sequence because it is reported to have a high DNA binding strength which leads to strong nucleosome positioning and therefore facilitates nucleosome assembly and stability. The histone binding and nucleosome formation affinity of the Widom sequence is sixfold or more greater than the affinity of even those natural DNA sequences with the strongest nucleosome positioning affinity (Thastrom et al; 1999). Other strong nucleosome binding DNA sequences include the 5S ribosomal RNA gene of the sea urchin Lytechinus variegatus (5S rRNA) and the long terminal repeat of the mouse mammary tumor virus (MMTV LTR) sequences (Flaus; 2012).

Recombinant nucleosomes comprising Widom 601 or other strongly nucleosome positioning DNA sequences are useful for many purposes. However, other novel purposes require an endogenous mammalian or human DNA sequence. We herein describe novel recombinant nucleosomes that include endogenous human DNA sequences, including cancer associated mutated DNA sequences, and novel applications for such nucleosomes.

### SUMMARY OF THE INVENTION

According to a first aspect of the invention, there is provided a method for assessing the performance of a DNA sequencing assay which detects a target endogenous target nucleotide sequence, comprising the steps of:
(a) adding a known concentration of a control material to a sample, wherein the control material comprises a recombinant nucleosome comprising the target endogenous nucleotide sequence or a fragment thereof; and
(b) performing the DNA sequencing assay on the sample prepared in step (a).

According to a further aspect of the invention, there is provided a method for the analysis of DNA in a sample, wherein said analysis is calibrated using a recombinant nucleosome comprising a target endogenous nucleotide sequence, optionally wherein the target endogenous nucleotide sequence comprises a mutation associated with increased risk of cancer.

According to a further aspect of the invention, there is provided a recombinant nucleosome comprising an endogenous nucleotide sequence, optionally wherein the endogenous nucleotide sequence comprises a mutation associated with increased risk of cancer.

### BRIEF DESCRIPTION OF THE FIGURES

FIG. 1: Diagram of DNA test steps controlled by DNA or nucleosome reference materials. Illustration of the major DNA test steps that may be controlled by use of nucleic acid (DNA) based or nucleosome based reference materials.

FIG. 2: Electrophoresis gel results for mutated BRAF 147 bp-nucleosomes. Lane 1: Free 147 bp BRAF oligonucleotide; Lane 2: Ladder; Lanes 3 and 4: Recombinant nucleosomes comprising 147 bp BRAF oligonucleotide.

FIG. 3: Electrophoresis gel results for mutated EGFR 147 bp-nucleosomes. Lane 1: Free 147 bp EGFR oligonucleotide; Lane 2: Ladder; Lanes 3 and 4: Recombinant nucleosomes comprising 147 bp EGFR oligonucleotide.

FIG. 4: Electrophoresis gel results for mutated KRAS 147 bp-nucleosomes. Lane 1: Free 147 bp KRAS oligonucleotide; Lane 2: Ladder; Lanes 3 and 4: Recombinant nucleosomes comprising 147 bp KRAS oligonucleotide.

FIG. 5: Immunoassay results obtained for recombinant human nucleosomes containing 147 bp mutated human BRAF, EGFR or KRAS DNA sequences. Well characterised recombinant H3.1-nucleosomes containing a Widom sequence were tested at concentrations of 1000 ng/ml, 100 ng/ml and 10 ng/ml. In addition, serial 1:10 dilutions of mutated human KRAS, BRAF or EGFR 147 bp-nucleosome preparations were tested.

FIG. 6: Analysis of spiked recombinant nucleosome reference material added to plasma by Next Generation Sequencing (NGS)

Known quantities of KRAS recombinant nucleosomes and Hela cell derived mono-nucleosomes were diluted into (A) artificial plasma and (B) human donor EDTA plasma in various proportions, and analysed for allelic frequency (AF) by NGS. The AF determined by NGS agreed well with the theoretical AF predicted by nucleosome mixing ratios.

FIG. 7: Analysis of spiked recombinant nucleosome reference material added to plasma by Bioanalyzer

Known quantities of KRAS recombinant nucleosomes and Hela cell derived mono-nucleosomes were diluted into a human plasma sample that contained larger endogenous (likely NETs derived) chromatin in various proportions, and analysed for DNA fragment size distribution using an Agilent Bioanalyzer. The results showed an expected single narrow peak for the recombinant KRAS nucleosomes, as well as a more diffuse mono-nucleosome peak for Hela cell derived nucleosomes at approximately 130-200 bp and another peak for endogenous larger oligo-nucleosome material at approximately 700-10,000 bp size range.

### DETAILED DESCRIPTION

### DNA Assays and Liquid Biopsy

The majority of DNA sequencing assays are performed on tissue samples or cellular samples. The research or clinical objectives of these assays may be many and varied. In these assays, DNA is extracted from cells or from tissue samples and sequenced. One area of medicine in which tissue or cellular DNA testing is commonly used is in oncology.

DNA abnormalities are characteristic of cancer diseases. The DNA of cancer cells differs from that of healthy cells in many ways including, but not limited to, point mutations, translocations, gene copy number, micro-satellite abnormalities, DNA strand integrity and nucleotide modifications (for example methylation of cytosine at position 5). These tumor-associated-alterations in DNA structure or sequence are investigated routinely in cancer cells or tissue removed at biopsy or surgery for clinical diagnostic, prognostic and treatment selection purposes.

However, tissue or cellular sample materials have limitations for DNA testing in oncology. Tumor genetic and epigenetic characteristics vary between different tumor types and between different patients with the same tumor disease. Moreover, these characteristics vary over time within the same cancer of the same patient with the progression of the disease and in the development of acquired resistance to drug or other therapies. Thus, serial investigation of tumor DNA in cells removed at surgery or biopsy may help the clinician to assess minimal residual disease, predict patient prognosis, select appropriate treatments for the patient, monitor disease progression and detect any relapse or acquired treatment resistance at an early stage (possibly many months earlier than radiological detection) and allow potentially successful changes in treatment courses.

Tissue DNA tests involve tissue biopsy procedures which cannot be performed repeatedly on patients. For some patients, tissue biopsy may not be used at all. Tissue biopsy is expensive to perform, uncomfortable for the patient, poses patient risk, and may lead to surgical complications. Moreover, a tumor in a patient may consist of multiple tumoral clones located within different areas of the same tumor or within different metastases (in metastatic cancer) not all of which may be sampled by biopsy. A tissue biopsy DNA investigation therefore provides a snap-shot of the tumor, both in time and in space, amongst different tumor clones located within different areas of a tumor at one particular moment in time.

The blood of cancer patients contains circulating tumor DNA (ctDNA) which is thought to originate from the release of chromatin fragments or nucleosomes into the circulation from dying or dead cancer cells. Tumor derived ctDNA circulates as small DNA fragments consistent with the size expected for mononucleosomes. Investigation of matched blood and tissue samples from cancer patients shows that cancer associated mutations, present in a patient's tumor (but not in his/her healthy cells) can also be present in ctDNA in blood samples taken from the same patient (Newman et al, 2014). Similarly, DNA sequences that are differentially methylated (epigenetically altered by methylation of cytosine residues) in normal and cancer cells can also be detected as methylated sequences in ctDNA in the circulation. In addition, the proportion of cell-free circulating DNA (cfDNA) that is comprised of ctDNA is related to tumor burden (McEvoy et al; 2018), so disease progression may be monitored both quantitatively by the proportion of ctDNA present and qualitatively by its genetic and/or epigenetic composition. Analysis of ctDNA can produce highly useful and clinically accurate data pertaining to DNA originating from all or many different clones within the tumor and which integrates the tumor clones spatially. Moreover, repeated sampling over time is a much more practical and economic option. Analysis of ctDNA has the potential to revolutionize the detection and monitoring of tumors, as well as the detection of relapse and acquired drug resistance at an early stage for selection of treatments for tumors through the investigation of tumor DNA without invasive tissue biopsy procedures. Such ctDNA tests may be used to investigate all types of cancer associated DNA abnormalities (e.g. point mutations, nucleotide modification status, translocations, gene copy number, micro-satellite abnormalities and DNA strand integrity) and would have applicability for routine cancer screening, regular and more frequent monitoring and regular checking of optimal treatment regimens (Zhou et al, 2012).

Cell free tumor DNA in the form of nucleosomes may be found in a variety of body fluids including sputum, urine, stool, saliva, Bronchial Alveolar Lavage and others and any of these may be used as a substrate material for DNA assays requiring standardisation and control. Circulating tumor DNA (ctDNA) occurs in blood, plasma or serum and any of these may be used as a substrate for ctDNA assays requiring standardisation and control. Any DNA analysis method may be employed including, without limitation, genetic DNA sequencing, epigenetic DNA sequencing analysis (e.g. for sequences containing 5-methylcytosine, 5-hydroxymethylcytosine), PCR, BEAMing, Next Generation Sequencing (NGS) (targeted or whole genome), digital PCR, isothermal DNA amplification, cold PCR (co-amplification at lower denaturation temperature-PCR), MAP (MIDI-Activated Pyrophosphorolysis), PARE (personalized analysis of rearranged ends) and Mass Spectrometry.

DNA analysis may include analysis for any genetic DNA markers including nucleotide substitutions, nucleotide insertions, nucleotide deletions, methylated DNA sequences or other DNA sequence mutations. Typical cancer associated DNA abnormalities that may be investigated in such an analysis include, without limitation, point mutations, translocations, gene copy number mutations, microsatellite abnormalities, DNA strand integrity and gene methylation status.

DNA analysis may involve determining the mutant allele fraction (MAF), i.e. the proportion of alleles at a specific genomic location which are mutant. MAF is generally expressed as a fraction or a percentage.

A large number of genes have been found to be mutated in cancer patients generally but any particular mutation is likely to occur in only a small proportion of cancer patients. However, all cancers patients do have some mutations, so the probability of detecting at least one mutation in any particular patient increases with the number of potential mutations tested. For this reason, it is usual to test for a panel of genetic mutations. Such a panel might, without limitation, include one or more mutations in the ABL1, ACVR1, ACVR1B, ACVR2A, AJUBA, AKT1, AKT2, AKT3, ALB, ALK, AMER1, APC, APEX1, APLNR, APOB, AR, ARAF, ARHGAP35, ARID1A, ARID2, AR/D5B, ATF71P, ATM, ATP11B, ATR, ATRX, ATXN3, AURKA, AX/N1, AX/N2, B2M, BAP1, BCL2, BCL2L1, BCL2L11, BCL9, BCOR, BIRC2, BIRC3, BRAF, BRCA1, BRCA2, BRD7, BTG2, BTK, CARD11, CASP8, CBL, CCND1, CCND2, CCND3, CCNE1, CD44, CD70, CD79B, CDH1, CHD3, CHD8, CDK12, CDK2, CDK4, CDK6, CDKN2A, CDKN2B, CEBPA, CHD4, CHEK2, COL5A1, CREBBP, CSF1R, CSNK2A1, CTNNB1, CTNND1, CUL1, CUL3, CYP2C19, CYP2D6, DACH1, DCUN1D1, DDR2, DICERI, DNMT3A, DPYD, EEF2, EGFR, ELF3, EP300, EPHA2, EPHA3, EPHA5, ERBB2, ERBB3, ERBB4, ERCC2, ESR1, EZH2, FANCA, FANCC, FANCD2, FANCE, FANCF, FANCI, FANCL, FAS, FAT1, FBXW7, FGF3, FGFR1, FGFR2, FGFR3, FGFR4, FLCN, FLT1, FLT3, FLT4, FOXA1, FOXA2, FOXQ1, GAS6-AS1, GATA1, GATA2, GATA3, GATA6, GNA11, GNAQ, GNAS, H3F3A, H3F3C, HGF, HIST1H3B, HNF1A, HRAS, IDH1, IDH2, IGF1R, IL6, IL6ST, IL7R, INSR, JAK1, JAK2, JAK3, KDM6A, KDR, KEAP1, KIT, KNSTRN, KRAS, KMT2A, KMT2B, KMT2C, KMT2D, LYN, MAGOH, MAP2K1, MAP2K2, MAP2K4, MAP3K1, MAP3K4, MAPK1, MDM2, MDM4, MECOM, MED12, MEN1, MET, MGA, MLH1, MPL, MRE11A, MSH2, MSH3, MSH6, MTOR, MUC6, MYC, MYCL, MYCN, MYD88, MYO18A, NCOR1, NF1, NF2, NFE2L2, NKX2-1, NKX2-8, NOTCH1, NOTCH2, NOTCH3, NPM1, NRAS, NSD1, NTRK1, NTRK2, NTRK3, NUP133, NUP93, PALB2, PAX5, PBRM1, PD-1, PDGFRA, PDGFRB, PD-L1, PD-L2, PK3CA, PK3CB, PK3CG, PK3R1, PK3R2, P/M1, POLD1, POLE, PPP2R1A, PPP6C, PRKAR1A, PRKCI, PRKDC, PSIP1, PMS2, PTCH1, PTEN, PTMA, PTPDC1, PTPN11, PTPRC, PTPRD, RAC1, RAD21, RAD50, RAD51, RAD51B, RAD51C, RAD51D, RAF1, RARA, RASA1, RBI, RBM10, RET, RFC1, RHEB, RHOA, RHOB, RICTOR, RNF43, ROS1, RPS6KA3, RPS6KB1, RPTOR, RQCD1, RRAS2, RUNX1, RUNX1T1, RXRA, SCAF4, SETBP1, SETD2, SF1, SF3B1, SIN3A, SLX4, SMAD2, SMAD4, SMARCA1, SMARCA4, SMARCB1, SMC1A, SMC3, SMO, SOS1, SOX17, SOX2, SOX9, SPOP, SPTA1, SPTAN1, SRC, SRSF2, STAG2, STAT3, STK11, TAF1, TBL1XR1, TBX3, TCEB1, TCF12, TCF7L2, TET2, TGFBR2, TGIF1, THRAP3, TLR4, TMSB4X, TNFAIP3, TOP1, TOP2A, TP53, TPMT, TRAF3, TSC1, TSC2, TSHR, TXNIP, U2AF1, UGT1A1, UNCX, USP9X, VHL, WHSC1, WTI XPO1 and ZFHX3 genes.

Similarly, many genes have been investigated as markers for differential cytosine methylation status in cancer. A few of these are SEPTIN-9, APC, DAPK, GSTP1, MGMT, P16, RASSF1A, TIG1, BRCA1, ERa, PRB, TMS1, MLH1, HLTF, CDKN2A, SOCS1, SOCS2, PAX5, PGR, PTGS2 and RARβ2.

As DNA abnormalities are characteristic of all cancer diseases, ctDNA tests have potential applicability in all cancer diseases. Cancers investigated include, without limitation, cancer of the bladder, breast, colorectal, melanoma, ovary, prostate, lung liver, endometrial, ovarian, lymphoma, oral, leukaemias, head and neck, and osteosarcoma (Crowley et al, 2013; Zhou et al, 2012; Jung et al, 2010). The nature of ctDNA tests will now be illustrated by outlining some (non-limiting) example approaches.

The first example involves the detection of a cancer associated gene sequence mutation in ctDNA. Blood tests involving the detection of a single gene mutation in ctDNA generally have low clinical sensitivity. There are two reasons for this. Firstly, although all cancers have mutations, the frequency of any particular mutation in a particular cancer disease is usually low. For example, although KRAS and P53 mutations are regarded as two of the more frequent cancer mutations and have been studied in a wide range of cancers including bladder, breast, colon, lung, liver, pancreas, endometrial and ovarian cancers, they were detected in 23%-64% and 17%-54% of cancer tissue samples respectively. Secondly, even if the cancer tissue of a patient does contain the mutation, the level or concentration of mutated ctDNA present in the blood of the patient may be low and difficult to detect. For example, KRAS and P53 mutations could be detected in the ctDNA of 0%-75% of KRAS and P53 tissue positive patients. The sum of these two effects meant that KRAS or P53 mutations were detected in the blood of less than 40% of cancer patients (Jung et al, 2010).

A second example involves the detection of multiple cancer associated gene sequence mutations in ctDNA. Although mutations of any particular gene such as KRAS or P53 may be present in only a minority of cancers, all cancers contain mutations therefore a sufficiently large panel of mutations should in principle facilitate the detection of most or even all tumors. One way to increase the clinical sensitivity of such tests is therefore to test for a wide range of mutations in many genes. Newman et al. have taken this approach for non-small cell lung cancer (NSCLC) and investigated 521 exons and 13 intron sequences from 139 recurrently mutated genes. The mutations studied encompassed multiple classes of cancer associated genetic alterations, including single nucleotide variation (SNV) and fusion genes. In this way the authors reported the detection of more than 95% of stage II-IV tumors and 50% of stage I tumors with 96% specificity in ctDNA blood tests (Newman et al, 2014).

A third example involves the detection of cancer associated epigenetic alterations to particular gene sequences in ctDNA. This approach can be applied to any DNA or nucleotide modification. A prime example of this approach is the detection of genes which are differentially methylated at cytosine residues in certain cancers. A large number of genes have been investigated for this purpose in a variety of cancers. A few of these are SEPTIN-9, APC, DAPK, GSTP1, MGMT, P16, RASSF1A, T1G1, BRCA1, ERα, PRB, TMS1, MLH1, HLTF, CDKN2A, SOCS1, SOCS2, PAX5, PGR, PTGS2 and RARβ2 investigated in bladder, breast, colorectal, melanoma, ovarian and prostate cancers. Typically, bisulfite conversion sequencing methods are used in which DNA is extracted from plasma and then treated with bisulfite which converts unmodified cytosine residues to uracil. Sequencing, PCR or other methods can then be applied to determine whether a particular methylated gene sequence is present. An illustrative example of this approach is the detection of methylated SEPTIN-9 in ctDNA for the detection of Colorectal Cancer (CRC) which was reported to detect 48% of CRC cases with a clinical specificity of 91% (Church et al, 2014).

A fourth example involves analysis of multiple genomic methylation sites in cfDNA to provide a methylation pattern. This pattern can be compared to libraries of previously derived methylation patterns that are each unique to a certain tissue to identify cellular or tissue origin of the cfDNA as described for example in WO2019159184.

A fifth example is the “fragmentomics” approach involving sequence analysis of circulating DNA fragments and comparison with the results of nuclease-accessible site analysis (also known as DNase hypersensitivity analysis) or transposase accessible site analysis of tissues and cell lines. In this approach, the genome wide DNA protein occupancy pattern of any cell type may be established by nuclease digestion of the open (not protein bound) DNA in a cell.

Protein bound DNA is protected from nuclease digestion and, following extraction, may be sequenced to identify the unique DNA protein occupancy pattern (or unoccupied open DNA pattern) of a cell type. Circulating cfDNA fragments are similarly protected by protein binding which may be histone in nature, as in nucleosomes, or may be by other proteins such as transcription factors. The boundaries of cfDNA fragments relate to the binding to nucleosomes, transcription factors or other proteins and the fragmentation patterns obtained by sequencing an individual's cfDNA can be built into a map of nucleosome and other protein occupancy. Such cfDNA occupancy maps can be compared to the occupancy maps for known tissues or cancer cell lines derived as nuclease-accessible site maps. This method is reported to indicate that nucleosome spacing in regulatory elements and gene bodies, as revealed by cfDNA sequencing in healthy individuals, correlates strongly with the occupancy patterns of lymphoid and myeloid cell lines. Sequencing of cfDNA from late-stage cancer patients showed additional occupancy patterns that correlated most strongly with occupancy maps of cancer cell lines, often matching the anatomical origin of the patient's cancer (Snyder et al, 2016). This indicates that nucleosome and other protein occupancy analysis of cfDNA patterns may be used both to detect cancer and potentially to identify the organ site of the cancer.

A typical NGS ctDNA liquid biopsy method may be designed for many objectives including, without limitation, to determine the presence of a cancer, to determine minimal residual disease, to determine disease progression, to determine the tissue or organ affected by cancer, to select a treatment, to monitor the efficacy of a treatment and others.

The workflow of a typical NGS ctDNA liquid biopsy method may involve the steps of:
1. Obtaining a blood sample from a subject
2. Centrifuging the blood sample to produce a plasma sample. The plasma sample contains DNA fragments in the form cell free nucleosomes.
3. Extracting the DNA from the sample (including separation and extraction from the nucleosomes)
4. Preparing a DNA library (typically involving adapter ligation to DNA fragments)
5. Optionally amplifying the library
6. Sequencing the library (typically by NGS)
7. Analysing the sequence data produced using bioinformatics
8. Determining the result (according to the objective)

Other methods, for example methods employing nanopore sequencing, will vary.

In addition to oncology tests, liquid biopsy methods are used for tests in other areas of medicine including, without limitation, fetal medicine by assaying for fetal cfDNA in the maternal circulation and in organ transplantation by assaying for organ donor sequences in the circulation of an organ recipient.

### Standardisation of DNA Assays

Clinical oncology is being transformed by the adoption of NGS based diagnostics using tissue biopsy and liquid biopsy sample matrices. Liquid biopsy is increasingly used to select the best treatment options for cancer patients. Ensuring that DNA sequencing assay methods are standardised so that results produced in different laboratories are consistent and accurate is imperative given that a false negative or false positive result could cause a patient to be diverted from a most beneficial therapeutic option, and/or unnecessarily subjected to adverse drug effects. However, there is a lack of standardisation of NGS based diagnostic methods both in tissue and biopsy liquid. There are national, European and international societies devoted to making progress towards method standardisation including, for example, the International Liquid Biopsy Standardization Alliance (Connors et al; 2020). In large part, the lack of standardisation relates to a lack of agreed, well characterized and validated standard reference materials which makes standardisation, Internal Quality Control (IQC) and External Quality Assessment (EQA) of results (EQA) difficult.

There are well established methods and materials for the comparison and standardisation of diagnostic clinical tests for proteins and other analytes performed in different laboratories, using different assay methods produced by different manufacturers and possibly utilising different analytical methods. Standardisation of clinical assays requires broadly or universally recognised standard reference materials that can be analysed in different methods in different laboratories and/or at different times to ensure consistent results. Standard reference materials for small molecules such as glucose, urea, cholesterol etc are pure chemical preparations. Standard reference materials for protein analytes are normally recombinant proteins, because recombinant proteins can be prepared and tested as a single pure molecule with a defined absolute concentration. Biologically sourced proteins normally comprise a complex mixture of protein isoforms any of which may, or may not, be glycosylated at numerous different loci and to different extents. The mixture of protein molecules will inevitably produce different signals sizes in different assays. It is difficult to standardise different methods using reference materials that intrinsically produce different results in different assays.

Similarly, the accuracy of clinical tests is normally ensured by IQC and EQA of results. IQC typically involves testing quality control (QC) samples on a repeated (e.g. daily) basis to ensure the results produced are stable and consistent with those produced yesterday, last week, last year etc. EQA Schemes are typically run nationally or internationally by distributing quality control (QC) samples with an established result to many different laboratories to check all laboratories produce accurate results. Any laboratories producing unacceptable results undertake ameliorative measures to ensure correct results and optimal patient care. Biologically derived material, can be used in IQC and EQA samples to assess consistency or reproducibility but are less useful for determination of accuracy as this again requires standardised reference materials.

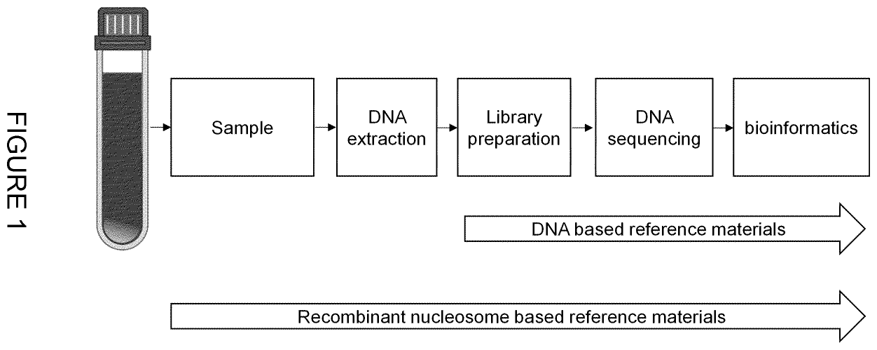

Current liquid biopsy reference materials are DNA fragments of known sequence. The DNA fragments may be spiked-in to samples to assess some steps of a liquid biopsy workflow. Referring to the steps 1-8 in the typical NGS ctDNA liquid biopsy workflow described above, such reference materials are suitable for the assessment of the reproducibility and accuracy of steps 4-8. Steps 1-3 cannot be assessed using DNA fragments because they involve nucleosomes and not DNA fragments. This is shown diagrammatically in FIG. 1, which illustrates that nucleic acid based reference materials can be used to control library preparation and subsequent assay steps, because these steps involve analyte nucleic acid material. However, nucleic acid reference materials cannot be used to control steps prior to library preparation, because these steps involve nucleoprotein, predominantly nucleosome, analyte material. Thus, the whole liquid biopsy workflow cannot be assessed and any error, variation or inconsistency in steps 1-3 will be missed.

It is clear that DNA fragments are suboptimal as reference material for liquid biopsy. None-the-less they are commonly used and available commercially from sources including SeraCare, Horizon and Sense-ID (Deveson et al; 2021).

To ensure clinical relevance the materials supplied are typically DNA fragments with relevant endogenous human DNA sequences including sequence mutations associated with cancer.

The DNA may be provided as a mixture of cancer derived and wild-type (healthy) DNA fragments to simulate patient samples with varying mutant allele fraction (MAF). In a typical example involving DNA fragments of endogenous human DNA gene sequence encoding the epidermal growth factor receptor (EGFR), for illustrative purposes only, an EGFR Multiplex cfDNA Reference Standard is available from Horizon Discovery to support the development and validation of cfDNA assays. The cfDNA materials are derived from human cell lines and fragmented to an average size of 160 bp to closely resemble cfDNA extracted from human plasma. The EGFR Multiplex cfDNA Reference Standard is supplied at 5%, 1%, 0.1% and 0% (EGFR Multiplex wild type) allelic frequencies and covers ten EGFR variants implicated in the responsiveness to EGFR tyrosine kinase inhibitors (EGFR-TKIs) and anti-EGFR monoclonal antibodies. The DNA fragments include clinically relevant SNPs, insertions and deletions in EGFR for liquid biopsy assay optimisation, validation and for routine monitoring of assay performance.

Chromatin is composed of nucleoprotein predominantly in the form of nucleosomes. The preanalytical steps involved in tissue or cfDNA assays, including the methods used for the extraction or isolation of DNA from a sample, are a critical component of tissue DNA or cfDNA analysis. It is clear that multiplex cfDNA reference molecules (provided as DNA) are poor reference or control materials for the standardisation of DNA assays because they are very different to the nucleoprotein chromatin matrix present in the sample. These cfDNA materials therefore provide a poor reference for control or standardisation of nucleosomes in the preanalytical steps of DNA assays, including the extraction of DNA from the nucleoprotein chromatin matrix in the sample.

The inventors have developed standardisation materials suitable for use with DNA sequencing assays. According to a first aspect of the invention, there is provided a method for assessing the performance of a DNA sequencing assay which detects a target endogenous nucleotide sequence, comprising the steps of:
(a) adding a known concentration of a control material to a sample, wherein the control material comprises a recombinant nucleosome comprising the target endogenous nucleotide sequence or a fragment thereof; and
(b) performing the DNA sequencing assay on the sample prepared in step (a).

As will be described herein, recombinant nucleosomes may be prepared containing the target of the DNA sequencing assay (i.e. a target endogenous nucleotide sequence). Nucleosomes containing endogenous sequences may be spiked into the assay to assess the performance (e.g., recovery, reliability, specificity, sensitivity) of the assay. In particular, the recombinant nucleosomes may be added before the samples are analyzed according to the user's routine protocol. The control material can be used by multiple laboratories, thereby increasing reliability of measurement of each targeted gene and increasing inter-experimental and inter-laboratory reproducibility of measurement.

References herein to a “control material” refer to means a material having a known and/or predetermined quantity or concentration of at least one, and possibly a plurality of, analyte(s) contained therein. The terms “standardisation material” or “reference material” may also be used.

In one embodiment, step (b) comprises the steps of:
(i) extracting DNA from the sample;
(ii) preparing a DNA library from the extracted DNA;
(iii) sequencing the DNA library; and
(iv) calculating the sequence coverage of the control material nucleotide sequence.

In a further embodiment, the method additionally comprises repeating steps (a) and (b) with a range of concentrations of the control material. The results may then be used to determine the sensitivity of the assay for detecting the mutation of interest.

In one embodiment, the DNA in the sample is cell free DNA (cfDNA). In a further embodiment, the cfDNA is circulating tumor DNA (ctDNA).

In one embodiment, the sample is a body fluid sample. The test samples to be sequenced may be any sample comprising cfDNA. In a preferred embodiment the test sample is a human or animal body fluid sample including for example a blood, serum, plasma, cerebrospinal fluid, urine, faeces, sputum or saliva sample. Blood, serum or plasma samples are of particular interest. Therefore, in one embodiment, the recombinant nucleosome is used to standardise a DNA sequencing assay performed in a blood, serum or plasma sample. Test samples may be prepared, for example where appropriate diluted or concentrated, and stored in the usual manner.

In a further embodiment, the body fluid sample is a blood, serum or plasma sample.

In one embodiment the DNA sequencing method is a liquid biopsy or cfDNA sequencing method. In another embodiment, the DNA sequencing method is a cell or tissue biopsy DNA sequencing method.

In one embodiment, the body fluid sample is from a healthy subject.

In an alternative embodiment, the body fluid sample is from a diseased subject.

In a further embodiment, the disease is cancer. As ctDNA and circulating nucleosomes are a feature of all cancer disease types investigated, the DNA sequencing method is for use in the detection and diagnosis of cancer. In one embodiment, the DNA sequencing method is for use in the detection of a cancer selected from: breast cancer, bladder cancer, colorectal cancer, skin cancer (such as melanoma), ovarian cancer, prostate cancer, gastric cancer, lung cancer, pancreatic cancer, bowel cancer, liver cancer, endometrial cancer, lymphoma, oral cancer, head and neck cancer, leukaemia and osteosarcoma.

References to “subject” or “patient” are used interchangeably herein. The subject may be a human or an animal subject. In one embodiment, the subject is a human. In one embodiment, the subject is a (non-human) animal. In one embodiment, the subject is a non-human mammal, such as a dog, cat, mouse, rat or horse, in particular a dog. The methods described herein may be performed in vitro or ex vivo.

It is possible that different nucleosomes with different epigenetic structures including different DNA modifications, histone modifications or histone isoforms may behave differently in DNA assay pre-analytics, for example with respect to DNA extraction from the nucleosomes. Therefore, in one embodiment of the invention, different nucleosomes containing different epigenetic structures are barcoded with different DNA sequences in addition to the endogenous or Widom sequences used. Thus, one barcode will be present on one nucleosome structure. This embodiment allows the standardisation of different nucleosome structures or other nucleoprotein structures in DNA assays by tracking the different nucleoprotein structures based solely on DNA sequencing at the end of the assay. The barcodes are designed such that they do not exist in the human or animal genome, avoiding any possibility of confusion with sample nucleosomes or cfDNA. Thus, the DNA from multiple nucleosome structures may be sequenced in a multiplex manner where each nucleosome type is identified by a unique barcode sequence.

### Recombinant Nucleosomes

References to “nucleosome” may refer to “cell free nucleosome” when detected in body fluid samples. It will be appreciated that the term cell free nucleosome throughout this document is intended to include any cell free chromatin fragment that includes one or more nucleosomes. In addition, a cell free nucleosome may be a mononucleosome (analogous to a single “bead”), an oligonucleosome (analogous to a string of “beads”), part of a larger chromatin fragment. Often the cell free nucleosomes present in a body fluid sample will be a mixture of some or all of these types.

It will be understood that a cell free nucleosome may be modified by modifying a component thereof. The term “component thereof” as used herein refers to a part of the nucleosome, i.e. the whole nucleosome does not need to be modified. The component of the cell free nucleosomes may be selected from the group consisting of: a histone protein (i.e. histone H1, H2A, H2B, H3 or H4), a histone post-translational modification, a histone isoform (also referred to herein as a histone variant), a protein bound to the nucleosome (i.e. a nucleosome-protein adduct), a DNA fragment associated with the nucleosome and/or a modified nucleotide associated with the nucleosome. For example, the component thereof may be histone (isoform) H3.1, histone H1 or DNA.

Recombinant nucleosomes are chemically synthesised nucleoproteins. Methods for the preparation of recombinant nucleosomes are known in the art and typically, involve the production of the individual recombinant core histones, assembling the individual histones into histone octamers and binding the octamers to suitable lengths of DNA to form recombinant nucleosomes (Dyer et al, 2004). Typically, recombinant nucleosomes are pure single molecular complexes comprising a single histone isoform combination. If additional modifications are to be included, e.g. post-translational histone modifications, these are purposely added and therefore are also uniform in terms of their histone modification composition. Thus, the recombinant nucleosomes represent a single molecule species, which is in contrast to biologically-derived nucleosomes which are a heterogeneous mix. In one embodiment, the present invention relates to the use of recombinant/semi-synthetic nucleosomes comprising an endogenous nucleotide sequence, wherein the endogenous nucleotide sequence comprises the mutation associated with an increased risk of cancer, for use as a standardisation material.

As used herein, a recombinant nucleosome (also called a designer nucleosome (dNuc)) is one that has been prepared by bringing together histones (including the core histones H2A, H2B, H3, and H4, and optionally linker histone H1), DNA, and optionally other factors to form the nucleosome. In other words, a recombinant nucleosome is one that is synthesized, not isolated from cells or chromatin. Each histone in the nucleosome may be independently fully synthetic, semi-synthetic (e.g., recombinantly produced and ligated to a synthetic peptide), or recombinantly produced. Each histone in the nucleosome may be a histone variant (e.g. H3.1, H3.3, H2A.Bbd, H2AZ.1, H2AZ.2, H2AX, mH2A1.1, mH2A1.2, mH2A2, or TH2B). The term recombinant nucleosome encompasses semi-synthetic nucleosomes and synthetic nucleosomes.

Recombinant nucleosomes have been synthesised to study their dynamics and for use as enzyme substrates, including for epigenetic regulators, and as calibration materials for immunochemical assays. Methods for the assembly of histones H2A, H2B, H3, H4 and DNA to form recombinant nucleosomes are known in the art. Typically, equimolar amounts of purified histones H2A, H2B, H3 and H4 are assembled to form a core histone octamer. A fragment of DNA is then bound around the histone octamer to form a nucleosome.

For current applications of recombinant nucleosomes, the nucleotide sequence of the DNA contained within the nucleosome is not critical. For this reason, most workers use a non-natural DNA sequence referred to as the Widom 601 sequence.

The nucleosome formation affinity of the Widom 601 DNA sequence is much greater than that of natural DNA sequences (Thastrom et al; 1999). This high affinity leads to strong nucleosome positioning and facilitates nucleosome assembly and nucleosome stability. As a specific DNA sequence incorporated into a nucleosome is not critical for nucleosome use as an enzyme substrate or for nucleosome immunochemical calibration purposes, workers in the field all typically use the Widom sequence.

A core nucleosome comprises approximately 147 bp DNA. However, most nucleosomes in vivo contain a further 20-80 bp of linker DNA (the DNA between nucleosomes). Similarly, cell free DNA (cfDNA) in plasma circulates in fragment sizes ranging between 120-220 bp, or multiples thereof, with a maximum peak at 167 bp corresponding to a mononucleosome including linker DNA. These mononucleosomes including linker DNA are therefore biological nucleosomes of interest.

It is clear that a DNA fragment that is greater than 147 bp in size may associate with a histone octamer in a variety of ways. For example, a histone octamer may associate with one (first) end of a 167 bp DNA fragment leaving approximately 20 bp of linker DNA at the other (second) end. Or the histone octamer may associate with the other, second, end of the 167 bp DNA fragment leaving approximately 20 bp of linker DNA at the first end. Or the histone octamer may associate with a sequence somewhere in the middle of the 167 bp DNA fragment leaving a length of linker DNA at both ends. In total there are approximately 40 different positions that may be occupied by a histone octamer on a 167 bp DNA fragment. Moreover, the octamer may not remain in one position but may move along the nucleosome and occupy different positions at different times. Clearly, this is not compatible with a stable, homogeneous recombinant nucleosome preparation.

Experimentally, nucleosomes have been observed to form at a set of preferred positions, rather than at a single predominant position. In many cases the most-preferred positions are related by intervals of multiples of 10 bp. This is reported to be a serious limitation for studies that would ideally be carried out using homogeneous nucleosome preparations (Lowary and Widom; 1998).

The double helix structure of DNA is characterised by one turn of the helix for every 10.5 bp of the DNA (Levitt; 1977). Similarly, nucleosome wrapping by DNA involves a 10.5 bp periodicity and cleavage of DNA by DNase yields a fragment size distribution with a 10 bp periodicity (Prunell; 1998). This also leads to a 10.5 bp periodicity in cfDNA fragment sizes (Snyder et al; 2016).

Widom used Fourier transform calculations to identify a special significance for nucleosome positioning of a motif consisting of about 10 bp periodic placement of TA dinucleotide steps. The original Widom 601 sequence is shown below. The nucleosome octamer core particle occupies the 147 bp span shown in upper case and flanking linker DNA at each end in lowercase.

Original Widom 601 sequence:
(SEQ ID NO: 1)
cgggatcctaatgaccaaggaaagcatgattcttcacaccgagtt
catcccttatgtgatggaccctatacgcggccgccCTGGAGAATC
CCGGTGCCGAGGCCGCTCAATTGGTCGTAGACAGCTCTAGCACCG
CTTAAACGCACGTACGCGCTGTCCCCCGCGTTTTAACCGCCAAGG
GGATTACTCCCTAGTCTCCAGGCACGTGTCAGATATATACATCCT
GTgcatgtattgaacagcgaccttgccggtgccagtcggatagtg
ttccgagctccc

Further amended Widom 601 sequences are known as well as other strongly nucleosome binding DNA sequences including the 5S rRNA and MMTV LTR sequences (see for example the sequences listed in Flaus; 2012).

The Widom 601 sequence has a high affinity for binding to histone octamers and therefore holds the octamer in a single nucleosome position. Moreover, its use facilitates nucleosome assembly and leads to more stable recombinant nucleosomes (Bouazoune et al; 2009). Widom 601 sequences are therefore used for the DNA fragment component of almost all recombinant nucleosomes regardless of the length of the component DNA fragment including nucleosomes comprising 147 bp DNA fragments.

### Recombinant Nucleosomes Comprising Endogenous Sequences

As discussed above, recombinant nucleosomes comprising Widom 601 or other strongly nucleosome positioning DNA sequences are useful for many purposes. However, other novel purposes require an endogenous mammalian or human DNA sequence. An ideal reference material for use in the standardisation of an assay should comprise known quantities of single defined molecules that are as similar as possible to the test material of the assay. In the case of cfDNA this would be defined nucleosomes in defined quantities that comprise DNA fragments of endogenous (human or other) sequences that may also include known cancer associated mutations.

Therefore, according to one aspect of the invention, there is provided a recombinant nucleosome comprising an endogenous nucleotide sequence, optionally wherein the endogenous nucleotide sequence comprises a mutation associated with increased risk of cancer.

In one embodiment, the endogenous nucleotide sequence is a mammalian nucleotide sequence.

In one embodiment, the endogenous nucleotide sequence is (or is derived from) an oncogene.

In one embodiment, the endogenous nucleotide sequence comprises a mutation associated with an increased risk with cancer.

Therefore, according to a particular embodiment of the invention, there is provided a method for assessing the performance of a DNA sequencing assay which detects a mutation associated with an increased risk of cancer, comprising the steps of:
(a) adding a known concentration of a control material to a sample, wherein the control material comprises a recombinant nucleosome comprising an endogenous nucleotide sequence, wherein the endogenous nucleotide sequence comprises the mutation associated with an increased risk of cancer; and
(b) performing the DNA sequencing assay on the sample prepared in step (a).

Acquired gene mutation is a major cause of cancer development and progression. BRAF, EGFR and KRAS are well known proto-oncogenes and have been major foci of oncology research. They are diagnostic and prognostic markers and are also specific targets in targeted therapies. Therefore, in one embodiment, the endogenous nucleotide sequence is derived from an oncogene. Examples of oncogenes include, without limitation, BRAF, KRAS, EGFR, SEPTIN-9, APC, DAPK, GSTP1, MGMT, P16, RASSF1A, T1G1, BRCA1, ERα, PRB, TMS1, MLH1, HLTF, CDKN2A, SOCS1, SOCS2, PAX5, PGR, PTGS2 and RARβ2.

These oncogenes present multiple mutations. Some example common mutations, without limitation, include:
BRAF V600E 1799 T>A: This mutation is the most common genetic alteration in melanoma, colorectal and papillary thyroid cancer. It is associated with aggressive disease and poor survival.
EGFR T790M 2369C>T: This mutation is used as a predictive biomarker for selection of non-small cell lung cancer (NSCLC) patients for treatment with EGFR-tyrosine kinase inhibitors (TKIs). TKI treatment has shown improved progression-free survival of patients with EGFR-mutated NSCLC. Examples include gefitinib, erlotinib, afatinib and dacomitinib.
KRAS G12D 35 G>A: This mutation is present in a variety of cancers including >30% of pancreatic cancer and 10% of colorectal cancer cases. Encouraging results have recently, been obtained for KRAS (G12C) inhibitors, such as AMG510 (sotorasib) and MRTX849 (adagrasib) in clinical trials (Huang et al, 2021).

The present inventors have demonstrated the preparation of stable, pure recombinant nucleosomes comprising an endogenous nucleotide sequence, wherein the endogenous nucleotide sequence is derived from the mutated BRAF(V600E), EGFR (T790M) or KRAS (G12D) genes. These results indicate that similar nucleosome products may be prepared for any gene of interest.

Therefore, in one embodiment the endogenous nucleotide sequence is derived from a gene selected from: BRAF, KRAS, EGFR, SEPTIN-9, APC, DAPK, GSTP1, MGMT, P16, RASSF1A, T1G1, BRCA1, ERα, PRB, TMS1, MLH1, HLTF, CDKN2A, SOCS1, SOCS2, PAX5, PGR, PTGS2 and RARβ2.

In a further embodiment, the endogenous nucleotide sequence is derived from a gene selected from: BRAF, KRAS or EGFR.

In a further embodiment, the mutation is BRAF V600E, KRAS G12D, or EGFR-T790M.

In a further embodiment, the endogenous nucleotide sequence comprises SEQ ID NO: 2, SEQ ID NO: 3, or SEQ ID NO: 4.

In one embodiment, the recombinant nucleosome additionally comprises a sequence that is not endogenous to a human, animal or other species. For example, a barcode nucleotide and/or a nucleotide sequence related to increased histone binding affinity.

Bar codes are useful in DNA sequencing based methods to facilitate the parallel simultaneous sequencing of DNA from multiple samples or sources. The DNA from different samples is labelled by attaching a short sample specific nucleotide sequence, or barcode, to each DNA molecule prior to pooling them into a mix containing a number of libraries to be sequenced simultaneously. After sequencing, the samples are binned by identifying the barcode sequence within each sequence read. Thus, the DNA from multiple patients may be sequenced in a multiplex manner where each patient is identified by a unique barcode sequence.

The DNA barcode may be any length and sequence that is suitable to uniquely identify the recombinant nucleosome. In some embodiments, the DNA barcode has a length of about 6 to about 50 bp, about 7 to about 30 bp, or about 8 to about 20 bp, for example, about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bp or any range therein.

The recombinant nucleosome may also additionally comprise a nucleotide sequence related to increased histone binding affinity, for example the Widom 601 sequence or a fragment or variant thereof.

Typically, DNA fragments of around 160 bp in length are used to mimic the size of typical cfDNA fragments comprising 147 bp of DNA plus some linker DNA. However, whilst typical cfDNA fragments are 160-170 bp in length, ctDNA fragments of tumor origin are typically shorter with a size range of approximately 90-200 bp with a peak at around the size associated with a mononucleosome containing no linker DNA. Indeed, 76% of ctDNA fragments of tumor origin are shorter than 150 bp in length (Mouliere et al; 2018). The present inventors therefore concluded that recombinant nucleosomes comprising DNA fragments of 147 bp in length are representative of typical tumor derived nucleosomes.

As previously mentioned, naturally occurring cell free nucleosomes comprise a range of ctDNA fragment sizes of approximately 90-200 bp in length (Mouliere et al; 2018). The authors reasoned that a cell free nucleosome in a subject may therefore exist in a constantly changing variety of nucleosome positioning structures. However, naturally occurring cell free nucleosomes containing DNA fragments of different sizes, must all be stable in solution in body fluids regardless of existing as a dynamic equilibrium mixture of nucleosome positioning structures available to them. If this were not the case they could not exist in the circulation. Therefore, recombinant nucleosomes of the invention are not limited by considerations of nucleosome positioning and may, like naturally occurring cell free nucleosomes, comprise a range of DNA oligonucleotide sizes.

A nucleosome of the invention may comprise a DNA fragment of any length. The oligonucleotide used may be shorter than 147 bp including, for example, an oligonucleotide comprising about 80 to about 145 bp in length, such as 80-99 bp, 90-99 bp, 100-110 bp, 111-120 bp, 121-130 bp, 131-140 bp or 141-146 bp. Similarly, a longer oligonucleotide may be used including, for example, an oligonucleotide comprising about 145 to about 300 bp in length, such as 148-150 bp, 151-160 bp, 161-170 bp, 171-180 bp, 181-190 bp, 191-200 bp, 201-220 bp, 221-230 bp, 231-240 bp, 241-250 bp, 251-260 bp, 261-270 bp, 271-280 bp, 281-290 bp or 291-300 bp. If a barcode sequence is present, this may be within the DNA sequence present in the recombinant nucleosome, i.e. within the 147 bp sequence. This means the barcode sequence is protected from modifying enzymes (such as MNase or Tn5) as it is located within the 147 bp sequence wrapped around the histones.

In one embodiment, a mixture of recombinant nucleosomes of the invention is provided that includes a range of DNA sizes to more closely mimic the range of sizes (rather than a single size) that occurs naturally. In one embodiment, a recombinant nucleosome of the invention is a di-nucleosome, tri-nucleosome or a polynucleosome.

The present inventors produced oligonucleotides comprising known endogenous human DNA sequences. It will be clear that DNA sequences of use in the methods of the invention may include other sequences, for example sequences that are similar, but not identical to, known sequences endogenous to humans or other species. Therefore, in a further aspect of the invention there is a provided a recombinant nucleosome comprising a DNA sequence that is similar to a sequence endogenous to humans or another species. For example, the DNA sequence may have greater than 50%, such as greater than 60%, 70%, 80%, 90%, 95%, 97% or 99% identity with a sequence endogenous to humans or another species.

### DNA Assay Calibrants

According to a further aspect of the invention, there is provided a method for the analysis of DNA in a sample, wherein said analysis is calibrated using a recombinant nucleosome comprising a target endogenous nucleotide sequence, optionally wherein the target endogenous nucleotide sequence comprises a mutation associated with increased risk of cancer.

In one embodiment, the analysis of DNA is to assess the content of ctDNA present in the sample. Therefore, in this context the recombinant nucleosome is used as a calibrant to measure the presence and/or concentration of the target ctDNA.

### Kits

According to a further aspect of the invention, there is provided a kit comprising a standardisation material and one or more reagents necessary to perform at least one of PCR, Multiplex-PCR and next-generation sequencing (NGS), wherein the standardisation material is a recombinant nucleosome comprising an endogenous nucleotide sequence. In a particular embodiment, the endogenous nucleotide sequence comprises a mutation associated with increased risk of cancer. The kit may be provided together with instructions for use of the kit in accordance with a DNA sequencing assay.

Kits of the invention may alternatively, or additionally, include reagents for the isolation and/or analysis of nucleosome associated DNA fragments. According to a further aspect of the invention, there is provided a kit which comprises: (i) a recombinant nucleosome of the present invention; and (ii) reagents for the isolation and/or analysis of nucleosome associated DNA fragments, optionally together with instructions for use of the kit in accordance with a DNA sequencing assay.

According to a further aspect, there is provided the use of a kit as defined herein for the diagnosis of cancer.

It will be understood that all embodiments described herein may be applied to all aspects of the invention.

Other features and advantages of the present invention will be apparent from the description provided herein. It should be understood, however, that the description and the specific examples while indicating preferred embodiments of the invention are given by way of illustration only, since various changes and modifications will become apparent to those skilled in the art.

The present invention will now be illustrated by the following examples.

### EXAMPLES

### Example 1

Recombinant histones were produced using standard techniques. Briefly, expression vectors were produced for each of histones H2A, H2B, H3 and H4. Recombinant histones were produced using vector transduced BL21 DE3 bacteria under isopropylthio-β-galactoside (IPTG) induction of transcription. As the bacteria store proteins produced in inclusion bodies, the bacteria were pelleted and sonicated to release the inclusion bodies. The recombinant histone proteins were released by incubation with guanidine hydrochloride buffer. Solubilized histone proteins were purified by reverse phase chromatography and the pure histone H2A, H2B, H3 and H4 preparations were aliquoted, lyophilized and stored at −20° C.

Histone core octamers were produced by rehydrating lyophilized histones in protein unfolding buffer. Recombinant histones were mixed together and dialysed to form the histone octamer. Histone octamer was purified by HPLC, concentrated using a centrifugation filter and stored at −20° C. with glycerol.

### Example 2

Plasmids bearing a mutated BRAF, EGFR or KRAS coding DNA sequence were used as templates for PCR amplification.

PCR primers (shown in Table 1) were designed to amplify a 147 bp BRAF, EGFR or KRAS sequence including the mutation of interest approximately in the middle of the sequence, taking the reading frame into account.

The resulting 147 bp sequences were as follows, with the mutation of interest highlighted in bold and underlined:

BRAF V600E sequence:
(SEQ ID NO: 2)
GACCTCAAGAGTAATAATATATTTCTTCATGAAGACCTCACAGTA
AAAATAGGTGATTTTGGTCTAGCTACAGAGAAATCTCGATGGAGT
GGGTCCCATCAGTTTGAACAGTTGTCTGGATCCATTTTGTGGATG
GCACCAGAAGTC
EGFR T790M sequence:
(SEQ ID NO: 3)
ATGGCCAGCGTGGACAACCCCCACGTGTGCCGCCTGCTGGGCATC
TGCCTCACCTCCACCGTGCAACTCATCATGCAGCTCATGCCCTTC
GGCTGCCTCCTGGACTATGTCCGGGAACACAAAGACAATATTGGC
TCCCAGTACCTG
KRAS G12D sequence:
(SEQ ID NO: 4)
ATGACTGAATATAAACTTGTGGTAGTTGGAGCTGATGGCGTAGGC
AAGAGTGCCTTGACGATACAGCTAATTCAGAATCATTTTGTGGAC
GAATATGATCCAACAATAGAGGATTCCTACAGGAAGCAAGTAGTA
ATTGATGGAGAA

TABLE 1
Forward (F) and reverse (R) PCR primer
sequences used to amplify the 147 bp
BRAF V600E, EGFR T790M and KRAS G12D
sequences.

| | | |
|---|---|---|
| BRAF | F | GACCTCAAGAGTAATAATATATTTCTTCATG |
| V600E | | AAGAC (SEQ ID NO: 5) |
| | R | GACTTCTGGTGCCATCCAC |
| | | (SEQ ID NO: 6) |
| EGFR | F | ATGGCCAGCGTGGACAAC |
| T790M | | (SEQ ID NO: 7) |
| | R | CAGGTACTGGGAGCCAATATTGTCTTTG |
| | | (SEQ ID NO: 8) |
| KRAS | F | ATGACTGAATATAAACTTGTGGTAGTTGGA |
| G12D | | (SEQ ID NO: 9) |
| | R | TTCTCCATCAATTACTACTTGCTTCCT |
| | | (SEQ ID NO: 10) |

The PCR product was pooled and purified by gel filtration. The purified DNA preparations were dried using a Speedvac system and sequenced by Sanger sequencing to check that the oligonucleotides had the expected sequences. The sequencing results matched perfectly with the expected sequences.

### Example 3

Nucleosomes comprising a histone octamer and mutated, cancer associated human DNA sequences were assembled directly by mixing the components in an appropriate ratio. Salt concentration dependent octamer assembly was performed by mixing histone octamer and 147 bp oligonucleotide in a high salt buffer and gradually reducing the salt level by dialysis filtration to induce assembly.

As a first step, various amounts of histone octamer were mixed with a fixed quantity of 147 bp DNA to establish the optimum assembly ratio for each DNA sequence. This ratio was then used for larger scale nucleosome preparation of mutated BRAF 147 bp-nucleosomes, mutated KRAS 147 bp-nucleosomes and mutated EGRF 147 bp-nucleosomes.

### Example 4

In order to demonstrate the assembly of the protein histone octamer with 147 bp mutated human cancer sequence oligonucleotides to form nucleosome complexes, we performed electrophoresis experiments on each of the three free 147 bp oligonucleotides comprising mutated BRAF, KRAS or EGRF DNA sequences, as well as on the mutated BRAF 147 bp-nucleosomes, mutated KRAS 147 bp-nucleosomes and mutated EGRF 147 bp-nucleosomes produced as described in EXAMPLES 1-3.

Briefly, 500 ng of nucleosome were loaded on 5% Tris-Borate-EDTA gel. Electrophoresis was performed at 100V for 1 hour at 2-8° C. The gel was stained with MIDORI green for 1 hour and visualized using a gel documentation tool.

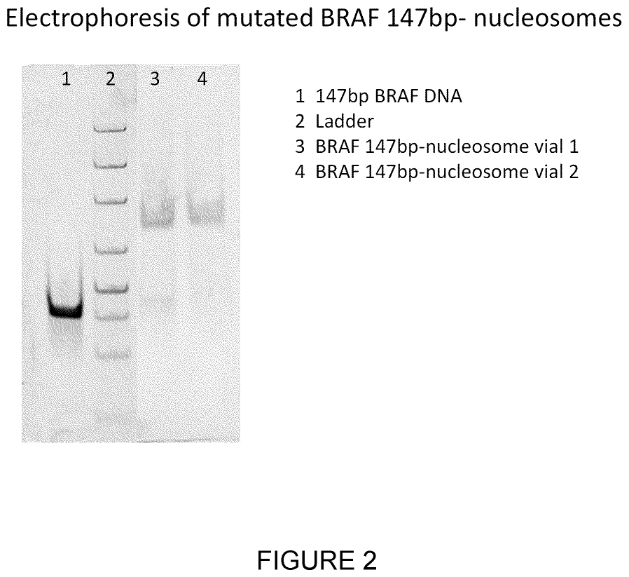

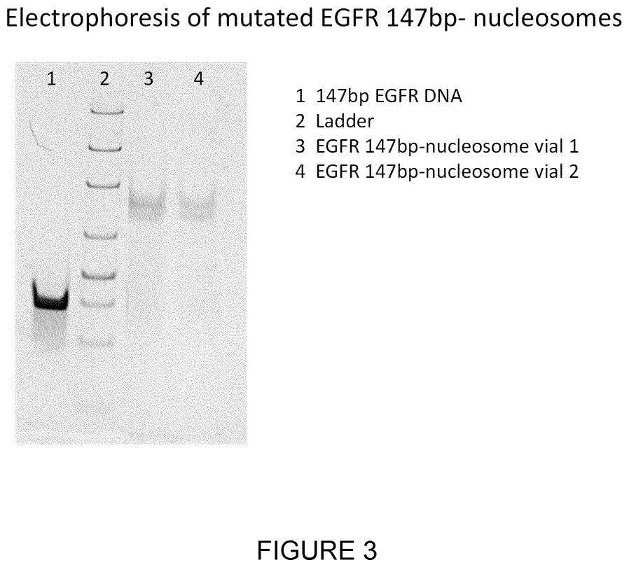

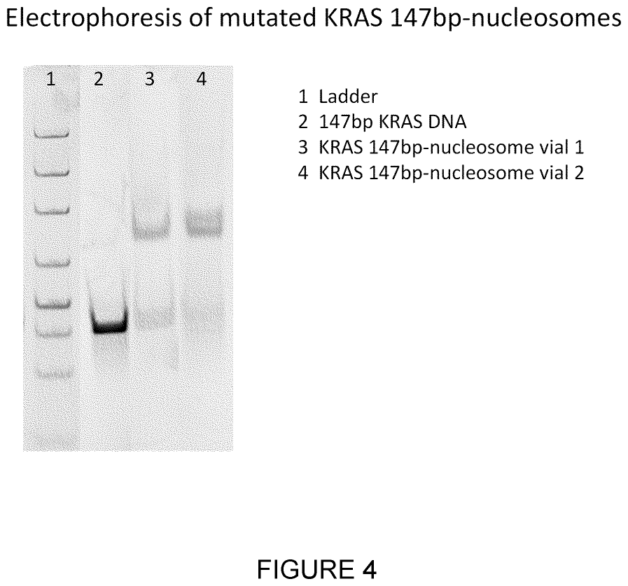

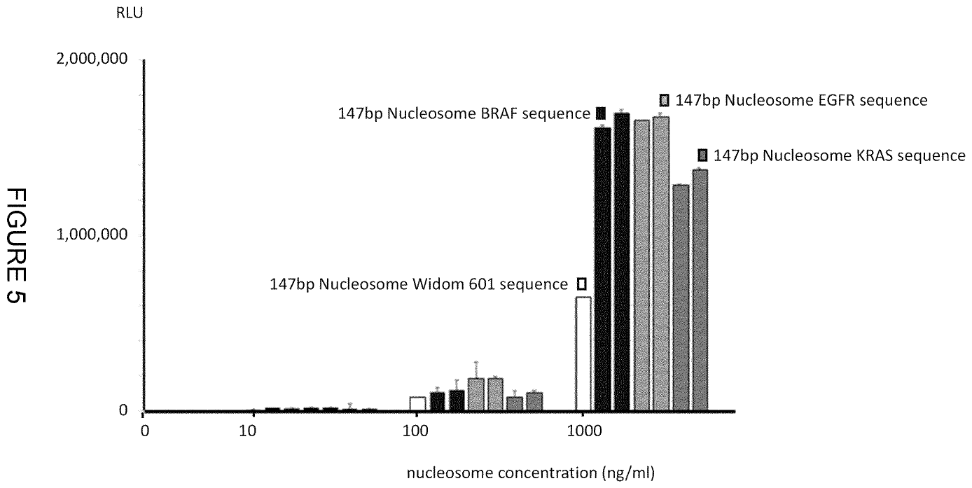

The results are shown in FIGS. 2, 3 and 4 for mutated BRAF 147 bp-nucleosomes, mutated KRAS 147 bp-nucleosomes and mutated EGRF 147 bp-nucleosomes, respectively. In each case the results showed that the free 147 bp DNA oligonucleotides migrated during electrophoresis and that nucleosome binding greatly diminished the migration. The results demonstrated the incorporation of DNA into a nucleosome format in each case.

### Example 5

We further confirmed the assembly of the protein histone octamer with 147 bp mutated human cancer sequence oligonucleotides to form nucleosome complexes, by testing the nucleosomes formed using an immunoassay which detects only intact nucleosomes (i.e. containing both a histone octamer and DNA). The assay employs one antibody directed to bind to a H3 protein present in the nucleosomes (histone H3.1) and one antibody directed to bind to a conformational nucleosome epitope present only in intact nucleosomes containing a histone octamer and DNA. The assay does not detect free histone octamer or free DNA.

Serial 1:10 dilutions of mutated human KRAS, BRAF or EGFR 147 bp-nucleosome preparations and well characterised recombinant H3.1-nucleosomes containing a Widom sequence oligonucleotide (i.e. control nucleosomes) at concentrations of 1000 ng/ml, 100 ng/ml and 10 ng/ml were tested. The assay used was an automated chemiluminescence immunoassay. Briefly, the nucleosome solutions (50 μl) were incubated with an acridinium ester labelled anti-nucleosome antibody (50 μl) and assay buffer (100 μl) for 1800 seconds at 37° C. Magnetic beads coated with an anti-histone H3.1 antibody (20 μl) were added, and the mixture was incubated a further 900 seconds. The magnetic beads were then isolated, washed 3 times and magnetic bound acridinium ester was determined by luminescence output over 7000 milliseconds.

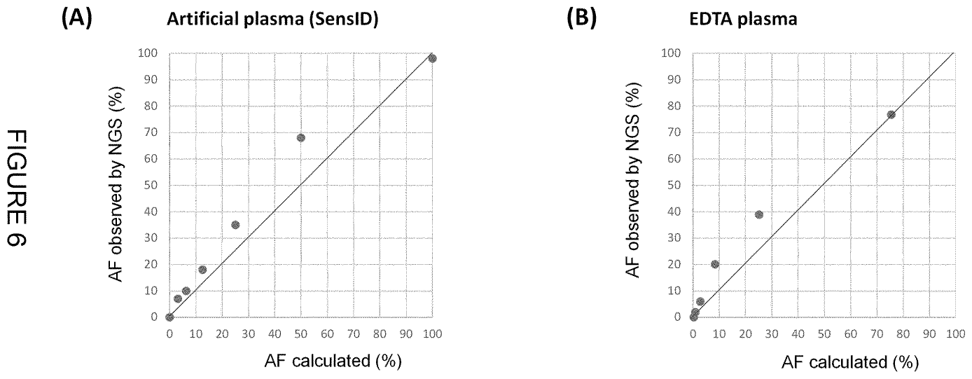

The results are shown in FIG. 5 and demonstrate that the mutated BRAF 147 bp-nucleosomes, mutated KRAS 147 bp-nucleosomes and mutated EGRF 147 bp-nucleosomes gave strong signals in the assay and were all intact nucleosomes containing both a histone octamer and DNA.

The 3 mutated BRAF, KRAS and EGFR gene sequences used for these examples were selected as gene mutations of interest in oncology. Stable, pure nucleosome preparations were prepared for all 3 gene sequences which indicates that similar nucleosome products may be prepared for any gene of interest.

### Example 6

Nucleosomes containing a mutated human oncogene sequence are produced as described in EXAMPLES 1, 2 and 3. A 187 bp sequence with a single point mutation is selected. One nucleosome is produced with a full 187 bp sequence oligonucleotide. One nucleosome is produced with a shortened 167 bp oligonucleotide. One nucleosome is produced with a further truncated 147 bp oligonucleotide. The 3 nucleosomes are characterised as described in EXAMPLES 4 and 5 and shown to be intact assembled nucleosomes.

### Example 7

An immunoassay recovery experiment is performed by spiking known amounts of recombinant nucleosomes containing mutated human DNA sequences into plasma samples.

The spiked samples (50 μl) are analysed by automated immunoassay to measure the resulting plasma levels of spiked nucleosomes. The analytical recovery of the nucleosomes is determined by comparing the quantity recovered versus the quantity added.

### Example 8

Recombinant nucleosomes containing mutated human DNA sequences are serially diluted to very low levels. Plasma samples obtained from healthy subjects and from patients diagnosed with a cancer disease are spiked at variable levels with the nucleosomes.

CfDNA is extracted from the spiked samples and DNA libraries for Next Generation Sequencing are prepared by standard methods used in the art. The sequencing results show a strong coverage of cfDNA fragments for the mutated sequences in samples containing higher levels of spiked nucleosomes but poor coverage for nucleosomes spiked at the lowest levels. The results are used to determine the analytical sensitivity of the assay and the performance of the ctDNA liquid biopsy assay.

The experiment is repeated using a variety of sample DNA extraction and library preparation methods to determine the most efficient sample treatment method for optimal ctDNA liquid biopsy analysis. The experiment is repeated in other laboratories to determine the relative analytical performance of ctDNA assays in different laboratories.

### Example 9

Recombinant nucleosomes containing mutated human DNA sequences are prepared as described in EXAMPLES 1-3 which additionally contain a DNA bar code sequence. A plasma spiking experiment is performed with the nucleosomes as described in EXAMPLE 8. The results are used for method optimisation within and between laboratories and for quality control of results within and between laboratory.

### Example 10

The results of procedures similar to those described in EXAMPLES 8 and 9 are used to standardise laboratory liquid biopsy methods within laboratories as well as between laboratories in different centres located across the world, leading to improved liquid biopsy performance world wide and improved patient outcomes.

### Example 11

To further demonstrate that the DNA and histones present in the nucleosome preparations described in EXAMPLES 1, 2 and 3 were comprised of intact nucleosomes, a chromatin immunoprecipitation (ChIP) experiment was performed. Briefly, nucleosomes bearing DNA of the Widom sequence or the mutated KRAS, BRAF or EGRF sequences were dissolved to make solutions of 1000 ng/ml. In principle, these solutions should contain only intact nucleosomes and no free DNA or free histone. An excess of anti-H3.1 nucleosome antibody attached to magnetic beads was added to each nucleosome solution. This action was expected to remove (by immunoprecipitation) all, or nearly all, intact recombinant nucleosomes from solution. Any DNA remaining in solution after ChIP may reflect the presence of free DNA (not nucleosome bound) in the recombinant nucleosome preparation.

DNA present in the solutions before and after ChIP was measured using a Bioanalyzer. The remaining DNA, after ChIP, as a percentage of that present before ChIP, was:
Widom: 1%
KRAS: 3%
BRAF: 2%
EGRF: 3%

The results indicate that at least 99%, 97%, 98% and 97% respectively of the DNA in the 4 solutions was present in the form of intact nucleosomes. The actual level of intact nucleosomes may be higher than these estimates as the efficiency of ChIP pull-down is less than 100%.

### Example 12

Nucleosomes bearing DNA of the mutated KRAS sequence were spiked into a commercially available (SensID) artificial/synthetic plasma and to the EDTA plasma of a healthy volunteer donor.

We added recombinant KRAS nucleosomes and Hela cell derived mono-nucleosomes to SensID synthetic plasma in different proportions to make a final concentration of 100 ng/ml in each combination. These were 100 ng/ml Hela nucleosomes and Ong/ml KRAS recombinant nucleosomes, 50 ng/ml Hela nucleosomes and 50 ng/ml KRAS recombinant nucleosomes, 25 ng/ml Hela nucleosomes and 75 ng/ml KRAS recombinant nucleosomes, 12.5 ng/ml Hela nucleosomes and 87.5 ng/ml KRAS recombinant nucleosomes, 6.25 ng/ml Hela nucleosomes and 93.75 ng/ml KRAS recombinant nucleosomes and Ong/ml Hela nucleosomes and 100 ng/ml KRAS recombinant nucleosomes. DNA was extracted from the samples and the allelic frequency (AF) of the KRAS sequence determined by DNA sequencing using Next Generation Sequencing. The results are shown in FIG. 6(A) and show that the sequencing results agree well with the theoretical AF of the nucleosome material input.

We performed a similar experiment using a human EDTA plasma sample collected from a healthy volunteer which contained endogenous nucleosomes that contributed 32.48 ng/ml to a final concentration of 132.48 ng/ml after spiking. As above, we added 100 ng/ml of a mixture of recombinant KRAS nucleosomes and Hela cell derived mono-nucleosomes to the human plasma in different proportions to make a final concentration of 132.48 ng/ml in each combination (rec. nucleosome+Hela nucleosome+endogenous nucleosome). DNA was extracted from the samples and the allelic frequency (AF) of the KRAS sequence determined by DNA sequencing using Next Generation Sequencing. The results are shown in FIG. 6(B) and show that the sequencing results agree well with the theoretical AF of the nucleosome material input.

These results show that recombinant nucleosomes of the invention behave similarly to, and are commutable, with natural endogenous mono-nucleosomes with respect to their analysis using NGS which is the most common liquid biopsy analysis method. Moreover, this is the case for their use both in plasma and in whole blood.

### Example 13

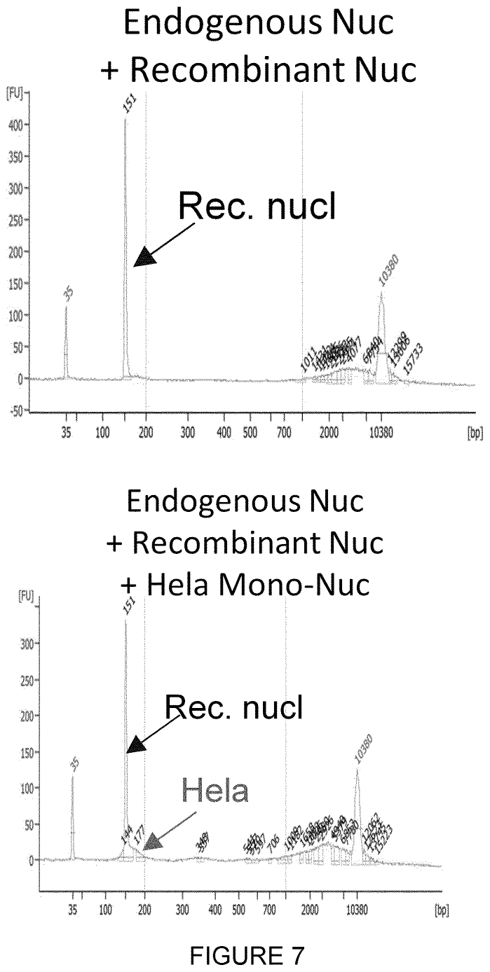

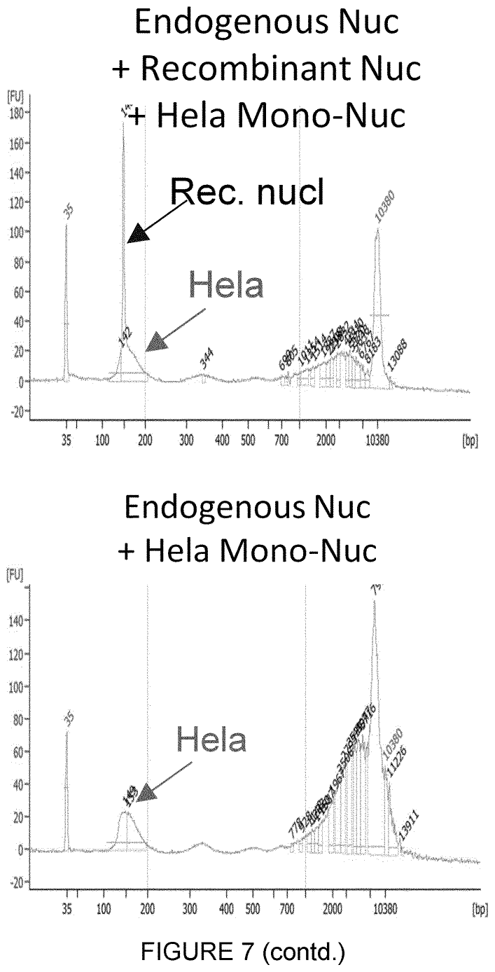

We performed a further experiment in which recombinant KRAS nucleosomes and Hela cell derived mono-nucleosomes were added to human whole blood that contained larger endogenous (likely NETs/genomic derived) chromatin to make a mixture that was 58% endogenous material and 48% spiked nucleosomes including varying proportions of KRAS recombinant nucleosomes and Hela cell derived nucleosomes. After plasma isolation, we analysed the DNA fragment profile of the nucleosome associated DNA by BioAnalyzer. The results showed a single narrow peak for the recombinant KRAS nucleosomes near to the expected size of 147 bp, a more diffuse mono-nucleosome peak for Hela cell derived nucleosomes at approximately 130-200 bp and another peak for endogenous larger oligo-nucleosome material at approximately 700-10,000 bp size range (FIG. 7). These results show that the recombinant nucleosomes of the invention behave similarly to, and are commutable with, biologically derived mono-nucleosomes.

### Example 14

The concentration of an EGFR nucleosome solution was measured by UV spectroscopy. The same material was also analysed by digital drop PCR. The concentrations measured by the two methods were the same within experimental error. Serial 10-fold dilutions of the EGFR nucleosomes (1, 1/10 and 1/100) gave linear results by digital drop PCR. These results indicate that reliable absolute concentration levels suitable for assignment of values for reference materials can be determined for recombinant nucleosomes of the invention.

### REFERENCES

Bouazoune et al. Nucleic Acids Research 37: 5279-94 (2009). doi.org/10.1093/nar/gkp524
Connors et al. Crit Rev Onc/Hem. 156: 103112 (2020). doi:1016/j.critrevonc.2020.103112
Church et al. Gut 63: 317-325 (2014).
Crowley et al. Nat. Rev. Clin. Oncol., 10(8): 472-484 (2013).
Deveson et al. Nat. Biotech. 39: 1115-28 (2021). doi:10.1038/s41587-021-00857-z.
Dyer et al, (2003) doi.org/10.1016/S0076-6879(03)75002-2
Flaus. Frontiers in Life Science. 5: 5-27 (2011). doi.org/10.1080/21553769.2012.702667
Herranz and Esteller. Methods Mol. Biol. 361: 25-62 (2007).
Huang et al. Sig. Transduct. Target Ther. 6: 386 (2021). doi.org/10.1038/s41392-021-2021
Jung et al. Clin. Chimica Acta., 411: 1611-1624, (2010)
Levitt. Proc. Natl. Acad. Sci. USA. 75: 640-644 (1977). doi.org/10.1073/pnas.75.2.640
Lowary and Widom. J M B. 19-42 (1998). doi.org/10.1006/jmbi.1997.1494
McEvoy et al. BMC Cancer 18: 726 (2018). doi.org/10.1186/s12885-018-4637-6
Mouliere et al. Sci. Transl. Med. 10: eaat4921 (2018)
Newman et al. Nat. Med., 20(5): 548-554 (2014)
Prunell. Biophysical Journal. 74: 2531-2544 (1988). doi:10.1016/S0006-3495(98)77961-5
Snyder et al. 2016. Cell 164: 57-68 (2016). doi.org/10.1016/j.cell.2015.11.050
Thastrom et al. JMB. 288: 213-229 (1999). doi.org/10.1006/jmbi.1999.2686 (Widom)
Van den Ackerveken et al. Sci Rep. 11: 7256 (2021). doi.org/10.1038/s41598-021-86630-3
Xiao et al. Genes and Development 19: 1444-1454 (2005). doi:10.1101/gad.1315905
Zhou et al. Semin. Oncol., 39(4): 440-448 (2012)

1. A method for assessing the performance of a DNA sequencing assay which detects a target endogenous nucleotide sequence, comprising the steps of:
(a) adding a known concentration of a control material to a sample, wherein the control material comprises a recombinant nucleosome comprising the target endogenous nucleotide sequence or a fragment thereof; and
(b) performing the DNA sequencing assay on the sample prepared in step (a).

2. The method of claim 1, wherein step (b) comprises the steps of:
(i) extracting DNA from the sample;
(ii) preparing a DNA library from the extracted DNA;
(iii) sequencing the DNA library; and
(iv) calculating the sequence coverage of the control material nucleotide sequence.

3. The method of claim 1 or claim 2, wherein the method additionally comprises repeating steps (a) and (b) with a range of concentrations of the control material.

4. The method of any of claims 1 to 3, wherein the DNA in the sample is cell free DNA (cfDNA).

5. The method of claim 4, wherein the cfDNA is all or partially circulating tumor DNA (ctDNA).

6. The method of any of claims 1 to 5, wherein the sample is a body fluid sample, such as blood, serum or plasma sample.

7. The method of any of claims 1 to 6, wherein the target endogenous nucleotide sequence is a mammalian nucleotide sequence.

8. The method of any of claims 1 to 7, wherein the body fluid sample is from a healthy subject.

9. The method of any of claims 1 to 7, wherein the body fluid sample is from a diseased subject.

10. The method of claim 9, wherein the disease is cancer.

11. The method of any of claims 1 to 10, wherein the target endogenous nucleotide sequence is an oncogene.

12. The method of any of claims 1 to 11, wherein the target endogenous nucleotide sequence comprises a mutation associated with an increased risk with cancer.

13. The method of any of claims 1 to 12, wherein the target endogenous nucleotide sequence is derived from a gene selected from: BRAF, KRAS, EGFR, SEPTIN-9, APC, DAPK, GSTP1, MGMT, P16, RASSF1A, T1G1, BRCA1, ERα, PRB, TMS1, MLH1, HLTF, CDKN2A, SOCS1, SOCS2, PAX5, PGR, PTGS2 and RARβ2.

14. The method of claim 13, wherein the target endogenous nucleotide sequence is derived from a gene selected from: BRAF, KRAS or EGFR.

15. The method of claim 14, wherein the mutation is BRAF V600E, KRAS G12D, or EGFR-T790M.

16. The method of any of claims 1 to 15, wherein the recombinant nucleosome comprises a DNA sequence that is 90 bp to 200 bp in length.

17. The method of claim 16, wherein the wherein the recombinant nucleosome comprises a DNA sequence that is less than 150 bp in length, such as 147 bp in length.

18. The method of any of claims 1 to 17, wherein the recombinant nucleosome additionally comprises a barcode nucleotide sequence.

19. The method of any of claims 1 to 18, wherein the recombinant nucleosome additionally comprises a nucleotide sequence related to increased histone binding affinity, such as the Widom 601 sequence (SEQ ID NO: 1) or a fragment or variant thereof.

20. A method for the analysis of DNA in a sample, wherein said analysis is calibrated using a recombinant nucleosome comprising a target endogenous nucleotide sequence, optionally wherein the target endogenous nucleotide sequence comprises a mutation associated with increased risk of cancer.

21. The method of claim 20, wherein the analysis of DNA is to assess the content of ctDNA present in the sample.

22. A recombinant nucleosome comprising an endogenous nucleotide sequence, optionally wherein the endogenous nucleotide sequence comprises a mutation associated with increased risk of cancer.

23. The recombinant nucleosome of claim 22, wherein the endogenous nucleotide sequence is derived from a gene selected from: BRAF, KRAS or EGFR.

24. The recombinant nucleosome of claim 23, wherein the mutation is BRAF V600E, KRAS G12D, or EGFR-T790M.

25. The recombinant nucleosome of claim 24, wherein the endogenous nucleotide sequence comprises SEQ ID NO: 2, SEQ ID NO: 3, or SEQ ID NO: 4.
